(19) United States
(12) Patent Application Publication
(10) Pub. No.: US 2025/0176817 A1
(43) Pub. Date: Jun. 5, 2025

(54) VIDEO LARYNGOSCOPE INTEGRATION WITH PATIENT ELECTRONIC MEDICAL RECORD

(71) Applicant: Covidien LP, Mansfield, MA (US)

(72) Inventors: Bo Chen, Louisville, CO (US); Peter Douglas Colin Inglis, Boulder, CO (US); Alexandra R. Hause, Denver, CO (US); Derek S. Tata, Longmont, CO (US)

(73) Assignee: Covidien LP, Mansfield, MA (US)

(21) Appl. No.: 18/963,134

(22) Filed: Nov. 27, 2024

Related U.S. Application Data
(60) Provisional application No. 63/605,359, filed on Dec. 1, 2023.
(60) Provisional application No. 63/649,074, filed on May 17, 2024.
(60) Provisional application No. 63/710,185, filed on Oct. 22, 2024.

(51) Int. Cl. A61B 1/267; A61B 1/00; A61B 1/04; G16H 10/60; G16H 30/40

(52) U.S. Cl. CPC A61B 1/267; A61B 1/00009; A61B 1/00016; A61B 1/0002; A61B 1/00045; A61B 1/04; G16H 10/60; G16H 30/40

(57) ABSTRACT

The technology relates to incorporating video laryngoscope data into the electronic medical record (EMR) of a patient. An example intubation system includes a patient terminal, a wireless hub, and a video laryngoscope. In an example method, the patient terminal accesses the electronic medical record of the patient; and receives, from the wireless hub wirelessly coupled to a video laryngoscope, intubation data generated by the video laryngoscope during an intubation procedure of a patient. The intubation data includes video data captured by the video laryngoscope and metadata for the video data generated by the video laryngoscope. The patient terminal then concurrently displays at least a portion of the intubation data with fillable fields of the electronic medical record; receives at least one interaction with the electronic medical record during the concurrent display; and stores the electronic medical record based on the at least one interaction.

### CROSS-REFERENCE TO RELATED APPLICATIONS

This application claims the benefit of U.S. Provisional Application No. 63/605,359 filed Dec. 1, 2023, entitled “Intubation Mode and Integration with Patient Electronic Medical Record,” U.S. Provisional Application No. 63/649,074 filed May 17, 2024, entitled “Intubation Mode and Integration with Patient Electronic Medical Record,” and U.S. Provisional Application No. 63/710,185 filed Oct. 22, 2024, entitled “Video Laryngoscope Integration with Patient Electronic Medical Record,” which applications are incorporated herein by reference in their entireties.

### BACKGROUND

Video laryngoscopes are commonly used to perform intubations on patients who require breathing assistance. During an intubation, the video laryngoscope may be used to manipulate the anatomy of the larynx and associated structures of a patient's airway, in order to obtain a view sufficient for insertion of a breathing tube (e.g., an endotracheal tube) into the trachea. Details of the intubation procedures, and the surgical procedures that follow, may need to be written into an electronic medical record (EMR).

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description, or may be learned by practice of the disclosure.

Aspects of the technology relate to integrating video laryngoscope data into a patient electronic medical record. In an aspect, the technology relates to a computer-implemented method for incorporating intubation data into an electronic medical record of a patient. The computer-implemented method includes accessing the electronic medical record of the patient. The method also includes receiving, from wireless hub wirelessly coupled to a video laryngoscope, intubation data generated by the video laryngoscope during an intubation procedure of a patient, where the intubation data includes video data captured by the video laryngoscope and metadata for the video data generated by the video laryngoscope. The method further includes concurrently displaying at least a portion of the intubation data with fillable fields of the electronic medical record. The method also includes receiving at least one interaction with the electronic medical record during the concurrent display, and storing the electronic medical record based on the at least one interaction.

In another aspect, the technology relates to a video laryngoscope. The video laryngoscope also includes a body; a display screen, a laryngoscope camera coupled to a distal end of the body, a processor. The laryngoscope also includes memory storing instructions that, when executed by the processor causes the video laryngoscope to perform operations may include: establish a wireless data connection with a wireless hub coupled to a patient terminal; capture, by the laryngoscope camera, image data during an intubation procedure; identify one or more anatomical features within the captured image data; identify one or more non-anatomical features within the captured image data; generating metadata, for the image data, based on the identified one or more anatomical features and the one or more non-anatomical features; generating an intubation data package may include the image data and the metadata; and transmitting, via the wireless data connection, the intubation data package to the wireless hub for incorporation into a patient electronic medical record at the patient terminal.

In another aspect, the technology relates to an intubation system the includes a video laryngoscope and a wireless hub. The video laryngoscope includes a laryngoscope camera; a first processor; and first memory storing instructions that, when executed by the first processor causes the video laryngoscope to perform operations. The operations include capture, by the laryngoscope camera, image data during an intubation procedure; identify one or more anatomical features or non-anatomical features within the captured image data; generate metadata, for the image data, based on the identified one or more anatomical features or non-anatomical features; and generate an intubation data package comprising the image data and the metadata. The wireless hub is in wireless communication with the video laryngoscope. The wireless hub includes a port coupled to a patient terminal; a second processor; and second memory storing instructions that, when executed by the second processor causes the wireless hub to perform operations. The operations include receive, from the video laryngoscope, the intubation data package; and transmit, via the port, the intubation data package to the patient terminal.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example anesthesia machine.

FIG. 2 depicts an example video laryngoscope.

FIG. 3A depicts an example view of a patient environment that includes a video laryngoscope in communication with a patient terminal.

FIG. 3B depicts an example view of an image captured by the video laryngoscope.

FIG. 3C depicts an example endotracheal tube.

FIG. 4 depicts an example schematic diagram of a video laryngoscope.

FIG. 5 depicts an example schematic diagram of an integrated electronic medical record (EMR) system.

FIGS. 6A-6B depict an example method for generating an intubation data package.

FIG. 6C depicts an example method for installing or activating software for viewing intubation data.

FIG. 7 depicts an example segment of an EMR record for intubation data.

FIG. 8 depicts a method for populating an electronic medical record with data associated with an intubation procedure.

### DETAILED DESCRIPTION

Patients who require breathing assistance may be connected to a mechanical ventilator via a breathing tube (e.g., an endotracheal tube). In a medical procedure referred to as an intubation, a clinician inserts a breathing tube into the mouth of the patient, past the larynx, and into the trachea. The breathing tube may then be connected to a ventilation system that includes an anesthesia machine, mechanical ventilator, and/or other device for supplying breathing gases (e.g., anesthetic gases, oxygen, etc.) to the patient.

A laryngoscope may be used during intubation to help the clinician manipulate portions of the patient's anatomy, such as the tongue and epiglottis, and obtain a view of the larynx sufficient for inserting the breathing tube into the trachea. To further help visualize the larynx, some laryngoscopes may be configured with a camera system that includes a video camera and light source. A laryngoscope that includes a camera system may be referred to as a video laryngoscope (VL). The video laryngoscope includes an integrated display on which the clinician may view images acquired by the camera.

A video laryngoscope may further include a feature that provides for wireless connection between the video laryngoscope and other medical devices or systems. For example, the video laryngoscope may establish connection with another medical device or computing device via an optical-based method (such as to pair with the device), and the video laryngoscope may then communicate with the other device using a more robust wireless communication method (e.g., WiFi or Bluetooth). One example pairing and transmission method is described in detail in U.S. Provisional Patent Application No. 63/505,275, titled Video Laryngoscope and Medical Device Wireless Video Transfer, which is incorporated herein by reference in its entirety.

Details of the intubation procedure may need to be incorporated into the patient's electronic medical record (EMR) after the intubation procedure or the following surgical procedure. Traditionally, data about any intubation procedure is manually entered by a clinician after the surgical procedure has been completed. This may lead potentially to inaccurate or incomplete information. In addition, the video data from the intubation procedure itself is generally not included in the EMR. In some cases, a clinician may take a photo with a separate device, such as a smartphone, of the screen of the video laryngoscope, and then incorporate that photo into the EMR as general image data. That general image data, however, has no tag or tie to the intubation procedure itself. Rather, there is simply a related-images category as part of the EMR, and those images may be related to any part of the procedure(s) performed on the patient. In still other examples, entering data into an EMR require a long path through multiple different devices, which requires significant security and authentication checks. For instance, a wired cord may be connected to a video laryngoscope monitor, and the monitor may download a video file. That video is then later uploaded to a cloud storage. A data security review occurs, and the video file may be associated manually with a patient, which may provide an opportunity for human or data-entry errors.

The technology disclosed herein, among other things, provides for integration of the video laryngoscope data into the current patient's EMR that provides for a more complete incorporation of video-laryngoscope data into the EMR along with additional insights and automated analyses of the video-laryngoscope data. For example, the video laryngoscope may be wirelessly connected to a VL-pairing device that is in communication with EMR device, such as a patient terminal that is used to populate the EMR for the patient. Through this wireless link, portions of the patient's EMR may be automatically populated with images, video, and/or other data from the video laryngoscope acquired or generated during the intubation procedure. Image processing of the video laryngoscope images and/or video may also be used to tag key points within the video-laryngoscope data, and such data may be incorporated into the EMR. Because the video-laryngoscope data is incorporated into the EMR via the patient terminal often while the patient is in the room and temporally proximate to when the intubation procedure was performed, there is also a higher confidence that the video-laryngoscope data is associated with the correct patient. Additional details are now provided via discussion of the included drawings.

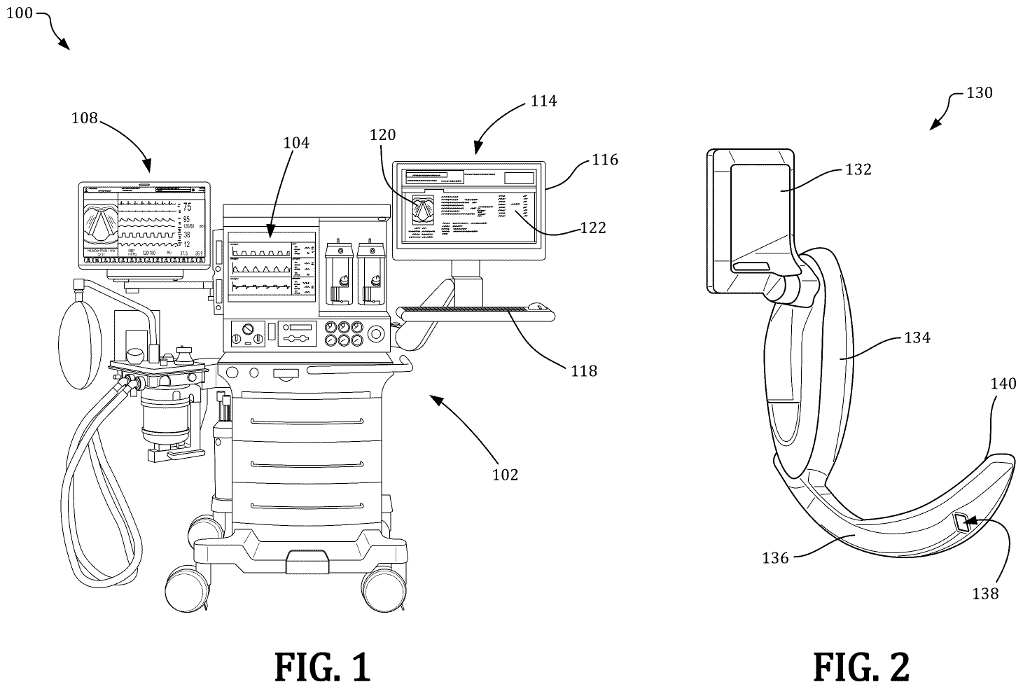

FIG. 1 depicts an example anesthesia machine 100 that includes a patient terminal 114 through which a patient EMR can be accessed and populated. While an anesthesia machine 100 is used as an example for which the patient terminal 114 may be attached, the patient terminal 114 may be stand-alone device and/or attached or integrated into another medical device.

The anesthesia machine 100 includes a base portion 102, which may include a cart and other primary controls of the anesthesia machine 100. In addition to the controls of the anesthesia machine 100, the base portion 102 may further include a primary display 104 that displays data related to the anesthesia operations of the anesthesia machine 100. In addition, the anesthesia machine 100 may include a secondary display or multi-parameter-monitor (MPM) display 108. During normal operation, the MPM display conveys patient parameters based on sensor data collected from various sensors that are connected to the anesthesia machine 100, such as a heart rate sensor, blood pressure sensor, pulse oximeter, capnometer, etc.

The patient terminal 114 may be coupled to, or included in, the anesthesia machine 100. The patient terminal 114 may be in the form of a computer that allows for interaction with the patient's EMR. For instance, the patient terminal 114 may include a terminal display 116 that display the EMR data and a terminal input device 118, such as a keyboard, mouse, touchscreen, etc.

The anesthesia machine 100 provides a supply of breathing gases (e.g., oxygen, nitrous oxide) mixed with a concentration of anesthetic vapor (e.g., isoflurane, sevoflurane). This mixture of gases is then delivered to the patient via a patient circuit that is connected to a patient interface (e.g., mask, endotracheal tube) of the patient. During an intubation procedure, a video laryngoscope is used to position the endotracheal tube (ETT) within the trachea of the patient. During at least part of this procedure, the patient circuit is not connected to the ETT or delivering the medical gases to the patient. For example, prior to intubation, the patient may be provided oxygen and anesthetic via a mask. During the intubation procedure, the mask is removed and the ETT is positioned in the patient with the guidance of the video laryngoscope. During that time, the patient is briefly disconnected from the anesthesia machine. As such, quickly and properly positioning the ETT can be a critical component of preparing the patient for a surgical procedure.

The data collected during the intubation procedure, such as by the video laryngoscope or the patient monitor 108, may then be used to populate the patient's EMR via the terminal display 116. For example, wireless transmission from the video laryngoscope may allow for the automatic population of data in the EMR entries for the intubation procedure. For instance, entries for the EMR may be completed through an Anesthesia Information Management System (AIMS) or similar system. AIMS may be considered a specialty form of EMR that is specifically designed for the unique needs of the anesthesia workflow. For instance, an example AIMS may be able to automatically capture data from the anesthesia machine and patient monitors, such as patient vitals, and populate that data into the EMR for the patient. Current EMR systems and AIMS technology, however, do not utilize video laryngoscope data for the population of the EMR.

With the technology disclosed herein, not only can the anesthesia information and patient parameters collected by the anesthesia machine 100 be automatically incorporated into the EMR, the video laryngoscope data captured during the intubation procedure may also be automatically incorporated into the EMR. For example, a copy of video file received for the intubation procedure from the video laryngoscope may be stored with the patient's EMR to allow for later access and review. Alternatively or additionally, the video data may be analyzed to extract data that is automatically incorporated into the EMR. For instance, object recognition algorithms may be executed against the video data to identify and/or characterize objects within the video. Such objects may include medical instruments (e.g., the ETT) and/or anatomical objects (e.g., vocal cords). Such extracted information is useful in populating the EMR portions that are directly related to the intubation procedure, such as fields relating to duration of the intubation procedure, size and type of ETT, laryngoscope blade type and size, use of stylets, use of a video laryngoscope, correct positioning confirmation data, number of attempts, etc. In some examples, this data may be populated into the fields automatically for review by the clinician and/or provided as suggestions to the clinician for data that may be populated within the field. In either case, the total manual entries required and total time to accurately complete the EMR is reduced significantly.

In some examples, the video-laryngoscope video data video data may be presented in an interactive manner concurrently with the fillable fields of the EMR. For instance, the terminal display 116 may display a video segment 120 and a field segment 122. The video segment 120 may include an interactive video captured by the video laryngoscope and/or data received by the anesthesia machine 100 during the intubation procedure. The clinician may be able to interact with the displayed video, such as to navigate to a particular point in time during the intubation procedure. In some examples, the video data may be analyzed to identify significant events during the intubation procedure, and the timestamps of those significant events may be tagged in the video file and presented via the video laryngoscope segment of the terminal display 116. For instance, markers or tags may be displayed on the navigation bar of the video playback interface that indicate when significant events occurred, such as a first detection of the ETT, a detection of the vocal cords, a detection that the tip of the ETT has passed through the vocal cords. Accordingly, the clinician may skip to particular time points of interest during the intubation procedure.

FIG. 2 depicts an example video laryngoscope 130. The video laryngoscope 130 includes a handle 134, a display 132, an extension arm 136, and a camera 138. A video laryngoscope blade 140 is also shown as attached to the video laryngoscope 130. When the blade 140 may be attached by sliding the blade 140 over the extension arm 136. The video laryngoscope 130 may be powered by a battery located in the handle 134.

When in use, the blade 140 and the arm 136 are positioned within the patient's mouth and upper airways. The camera 138 positioned at the end of the arm 136 captures images during the intubation procedure as the ETT is inserted past the camera 138 and the blade 140. The captured images may be captured as video. The live video feed may be presented on the display 132 of the video laryngoscope 130. The backside of the display 132 is depicted in FIG. 2, but the display of the video feed is presented on the front side of the display.

In some examples, the clinician may also interact with the video laryngoscope 130 to capture additional data about the intubation procedure. For instance, the video laryngoscope 130 includes one or more input elements, such as a button and/or the display 132 may be a touchscreen. The interactions may cause a certain point of the procedure to be marked or flagged as important. The interactions may also or alternatively cause a still image of the video feed to be captured and stored.

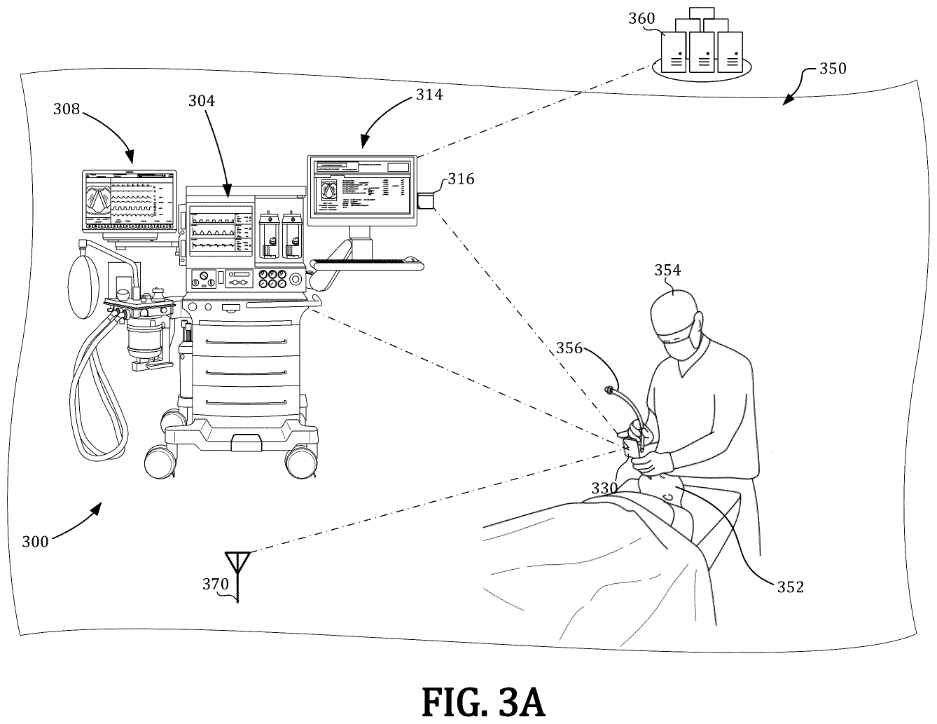

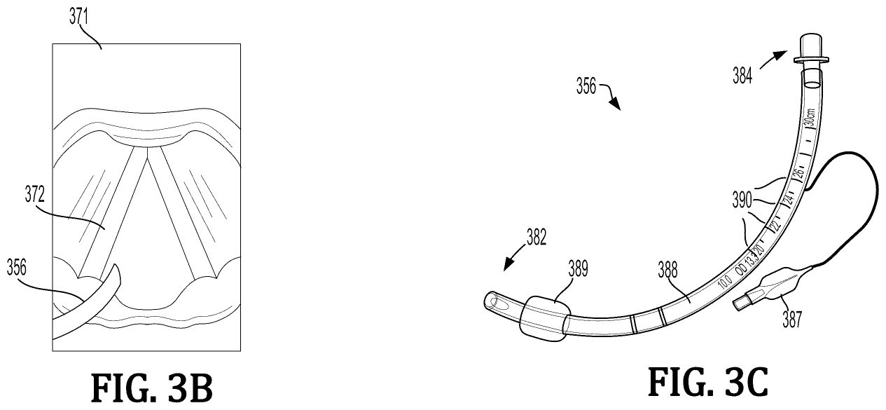

FIG. 3A depicts an example view of a patient environment 350 that includes an example video laryngoscope 330 in communication with an example patient terminal 314 of the anesthesia machine 300 during an intubation procedure. In the example depicted, the video laryngoscope 330 communicates with a wireless hub 316 that is connected to the patient terminal 314.

The patient environment 350 can be any room or theater where an intubation is being performed, such as a medical suite in a hospital or other care setting, an operating or other procedure room, patient recovery room, an emergency intubation setting (e.g., an ambulance), or other environments. The anesthesia machine 300 may be similar to, or the same as, anesthesia machine 100, depicted in FIG. 1. The video laryngoscope 330 may be similar to, or the same as, video laryngoscope 130, depicted in FIG. 2.

During the intubation procedure, the laryngoscope operator 354 (e.g., clinician) holds a handle of the video laryngoscope 330. Acquired image data is displayed on the display of the video laryngoscope 330. As part of an intubation procedure, the ETT 356 is advanced into the airway of a patient 352 to secure the airway for anesthesia. Accordingly, the operator 354 of the video laryngoscope 330 performs the intubation and directly manipulates the ETT within the patient's airway, and other clinicians in the patient environment assist the laryngoscope operator 354, monitor the condition of the patient 352, prepare or adjust medical equipment in the patient environment 350, and/or wait until the airway is secured to perform other procedures or interventions. As provided herein, the image data can be stored in a memory on the video laryngoscope 330. The image data may be in the form of video data (e.g., a video feed, video stream) and/or may be in the form of still images.

In the example depicted in FIG. 3A, the patient environment 350 includes an anesthesia machine 300, with which the video laryngoscope 330 may be in communication as described further herein. In addition, the anesthesia machine 300 may include, or be connected to, sensors for measuring physiological parameters of the patient during the intubation procedure, such as pulse oximetry sensors, heart rate sensors, ECG, sensors, capnometry sensors, blood pressure sensors, etc. Such data may be displayed on the MPM display 308 of the anesthesia machine 100. Some data may also be displayed on the primary display 304 of the anesthesia machine 300.

The video laryngoscope 330 wirelessly connects to patient terminal 314. Once connected, the video laryngoscope 330 transmits video image data to the patient terminal 314. In another example, VL-pairing components are incorporated into a portable module, such as a pluggable dongle, that can be connected to the patient terminal 314. One example of the pluggable module is the wireless hub 316. The video-laryngoscope data may then be transmitted from the video laryngoscope 330 to the wireless hub 316, which relays the video-laryngoscope data to the patient terminal 314 for incorporation into the current patient's EMR. The wireless hub 316 may be plugged into a port of the patient terminal 314, such as a universal serial bus (USB) port or other port of the patient terminal 314. In other examples, the wireless connection may be a direct connection between the patient terminal 314 and the video laryngoscope 330. For instance, the patient terminal 314 may include integrated components for wirelessly communicating with the video laryngoscope 330, as further discussed herein. In other examples, the wireless pairing and communication components for communicating with the video laryngoscope 330 may be integrated into another device that is then connected to the patient terminal 314. For example, the VL-pairing components may be incorporated into another component of the example anesthesia machine 300 and/or into the MPM, which subsequently communicates the video-laryngoscope data to the patient terminal 314.

As described in further detail below, connection or pairing of the video laryngoscope 330 with the video laryngoscope pairing components (e.g., the wireless hub 316) may be performed through the use of optical and non-optical signals with little to no input required from the laryngoscope operator 354 or other clinical staff. In an embodiment, when the video laryngoscope 330 is powered on (e.g., in response to a manual selection of a power button), the video laryngoscope 330 goes through an initial pairing process that includes the emission of an optical signal that is received VL-pairing components. For instance, an optical transceiver of the video laryngoscope 330 emits an optical signal through an optically transparent window of the video laryngoscope 330 such that the optical signal is emitted throughout the example patient environment 350. The VL-pairing components then detect the optical signal from the video laryngoscope 330. For example, the VL-pairing components may also include an optical transceiver. The optical transceiver of the VL-pairing components processes the received optical signal from the video laryngoscope 330, and the VL-pairing components then emit an optical response signal of its own via its optical transceiver. The video laryngoscope 330 receives this optical response signal, and a non-optical connection (e.g., WiFi, Bluetooth, etc.) between the video laryngoscope 330 and VL-pairing components may then be established.

Once the non-optical connection is established between the video laryngoscope 330 and VL-pairing components, the video image data (and/or still image data) captured by the video laryngoscope 330, is transmitted by the video laryngoscope 330 to VL-pairing components via the non-optical connection. Once received by the VL-pairing components and passed to the patient terminal 314, the video image data may be displayed and/or stored patient terminal 314, such as part of the EMR completion process.

The patient terminal 314 may include an exterior port for receiving the wireless hub 316 containing the VL-pairing components. In such examples, the wireless hub may be plugged directly into the patient terminal 314 and video data from the video laryngoscope 330 may be received by the patient terminal without the video data having to be transferred through or via other components of the anesthesia machine 300. In such examples, during the intubation procedure, the video data from the video laryngoscope 330 may be displayed on the patient terminal 314 alternatively (or in addition to) the MPM display 308.

In still other examples, the MPM display 308 may include an exterior port for receiving the wireless hub 316. In such examples, the video data from the video laryngoscope 330 may be received by the MPM display 308 without have to be transferred through or via other components of the anesthesia machine 300. During the intubation mode, the MPM display 308 accesses the video data received from the video laryngoscope 330, via the adapter, and displays the video data.

Once the patient EMR has been created and/or augmented via the patient terminal 314, the EMR may be communicated to one or more remote servers 360 for storage in such servers 360. The servers 360 are outside of the patient environment 350. For instance, the servers 360 may be on-premises of the hospital and/or cloud-based servers that are remote from the hospital.

In some examples, a locating beacon 370 may be present in the patient environment 350. The locating beacon 370 may emit wireless signals that are detected by the video laryngoscope 330, and/or the video laryngoscope 330 may emit wireless signals that are detected by the locating beacon 370. Based on the wireless signals, the locating beacon 370 and/or the video laryngoscope 330 detects that the video laryngoscope 330 is within the particular patient environment 350. The detection of being present within the patient environment 350 may include an identifier of the current patient environment 350 (e.g., particular room number, operating theater number or name). That identifier of the current patient environment 350 may then be included in the video-laryngoscope data that is transmitted to the patient terminal 314 and incorporated into the patient EMR. By including such an identifier (along with timestamp data) with the video-laryngoscope data, a later review of the EMR allows for a confirmation that the video-laryngoscope data is for the particular patient that was in the identified patient environment 350 at the time that the video-laryngoscope data was generated.

FIG. 3B depicts an example view of an image 371 captured by a camera of the video laryngoscope 330. The example image 371 includes an image of the vocal cords 372. The image 371 also includes a distal tip of the ETT 356. Anatomical items, such as the vocal cords 372, may be automatically detected in the images 371 by the technology disclosed herein. Non-anatomical instruments, such as the ETT 356 among other types of airway tubes, instruments, endoscopes, introducers, or the like, may also be automatically detected in the images 371 by the technology disclosed herein.

FIG. 3C depicts an example endotracheal tube (ETT) 356. When positioned in the airway of a patient, the ETT 356 may be used to pass breathing gases between the patient and a ventilator or anesthesia machine. As described above, during intubation the ETT distal end 382 is inserted through the mouth, past the larynx, and into the trachea of the patient. The ETT body 388 is a flexible tube that allows the ETT 356 to bend and flex as the ETT 356 is navigated into position. In some examples, the ETT body 388 may be shaped, such as with the curve depicted, to match the approximate shape of the airway, which may facilitate insertion of the ETT 356. The ETT body 388 may include depth markings 390 that provide an external visual indication of the insertion depth of the ETT 356. When positioned, a pilot balloon 387 may be used to inflate the cuff 389, which seals the ETT 356 against the walls of the trachea. The cuff 389 is shown inflated in FIG. 2A, but in some examples, the cuff 389 may remain uninflated during use.

In one example, the distal end of an endoscope or other type of endoluminal instrument may be inserted into the proximal opening of the ETT 356 and advanced through the the ETT 356. At the distal end 382 of the ETT 356, the distal tip of the endoscope may be advanced through the distal opening and into the airway. The endoscope may then provide video images of the airway, such as the trachea, carina, bronchi, and/or other structures of the airway and/or lungs of the patient.

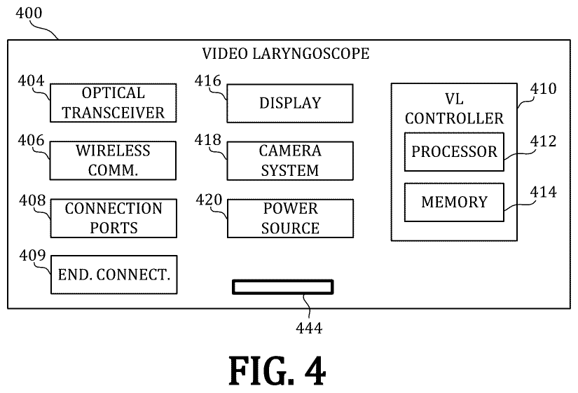

FIG. 4 depicts a schematic diagram of an example video laryngoscope 400, which may be the same as, or similar to, example video laryngoscope 130. The video laryngoscope 400 includes an optical transceiver 404 for transmitting and receiving the optical signals discussed herein. For example, the optical transceiver 404 may be a type of infrared (IR) transceiver or may be capable of transmitting and receiving optical signals in the visible wavelength. The video laryngoscope 400 includes an optical window 444 formed in the housing of the video laryngoscope 400 to allow for the optical signals to reach the optical transceiver 404 and exit the video laryngoscope 400.

The video laryngoscope 400 further includes a wireless communication device 406. The wireless communication device 406 may be a wireless transceiver that is configured to establish wireless communication in a non-optical frequency. By way of example, the wireless communication device 406 may be configured to communicate using the IEEE 802.15.4 standard, and may communicate, for example, using ZigBee, WirelessHART, or MiWi protocols. Additionally or alternatively, the wireless communication device 406 may be configured to communicate using the Bluetooth standard or one or more of the IEEE 802.11 standards or similar communication techniques. In some examples, the video laryngoscope 400 also include one or more connection ports 408. In examples, the connection ports 408 may include one or more external ports for establishing a wired connection.

In some examples, the video laryngoscope 400 may also receive images captured by an external endoscope camera that is connected to the video laryngoscope 400 via an endoscope port 409. Image data acquired by the external endoscope camera may be transmitted to the other devices discussed herein, such as the patient terminal, along with image data acquired by the video laryngoscope camera system 418.

The video laryngoscope 400 also includes a display 416, camera system 418, and power source (e.g., battery) 420. The camera system 418 includes a camera for imaging the patient's airway and a light source that illuminates the field-of-view (FOV) of the camera. The light source may be a type of LED, lamp, or other type of light-emitting element. The camera includes an imaging sensor, such as a charge-coupled device (CCD), complementary metal-oxide-semiconductor (CMOS), or other type of sensor.

The display 416 may be any of a variety of display technologies, such as liquid crystal display (LCD), light emitting diode (LED), organic light emitting diode (OLED), or other display technology. In examples, the display 416 may be a touch-sensitive display (e.g., a capacitive touch-sensitive display) capable of receiving input from a user. Aspects of the operation of the video laryngoscope 400 may also be configured via the display 416, such as video image display preferences and other configurable settings of the video laryngoscope 400. In one example, a clinician may tag or mark a video image by providing input via the display 416. For instance, a clinician may provide input through the display 416 to indicate a video segment of interest.

Video images acquired by the video laryngoscope camera system 418 may be displayed on the display 416 or may be combined with images acquired by an attached endoscope and displayed simultaneously. For example, the display 416 may be capable of providing split screen, picture-in-picture, or other method for simultaneously displaying video images.

The video laryngoscope 400 includes a controller 410 that includes one or more processors 412 and memory elements 414. The processor 412 may include one or more general purpose processors, microprocessors, microcontrollers, graphics processing units (GPUs), digital signal processors (DSPs), and/or other programmable circuits. In examples, the processor 412 may include any combination of commercially available components, and/or custom or semi-custom integrated circuits, such as application specific integrated circuits (ASICs). The processor 412 may include elements needed for control or communication with the display 416, camera system 418, wireless communication device 406, optical transceiver 404, and/or other elements of the video laryngoscope 400.

The processor 412 may perform control, interface, communication, or other processing functions by executing instructions that are stored in the memory 414. For instance, the memory 414 may store instructions that, when executed by the processor 412, cause the elements of the video laryngoscope 400 to perform operations described herein. In one example, the memory 414 may store portions of one or more algorithms associated with the analysis of the imaging data described below. In another example, the processor 412 and memory 414 may control the pairing process between the video laryngoscope 400 and the VL-pairing components. The memory 414 may include random access memory (RAM), read-only memory (ROM), electrically erasable programmable read-only memory (EEPROM), flash memory or other memory technology. The processor and memory may also perform the AI/ML functions (e.g., object detection in images) discussed herein. For instance, the trained AI/ML model(s) may be stored in the memory 414 of the video laryngoscope 400 and executed locally by the processor 412 of the video laryngoscope 400.

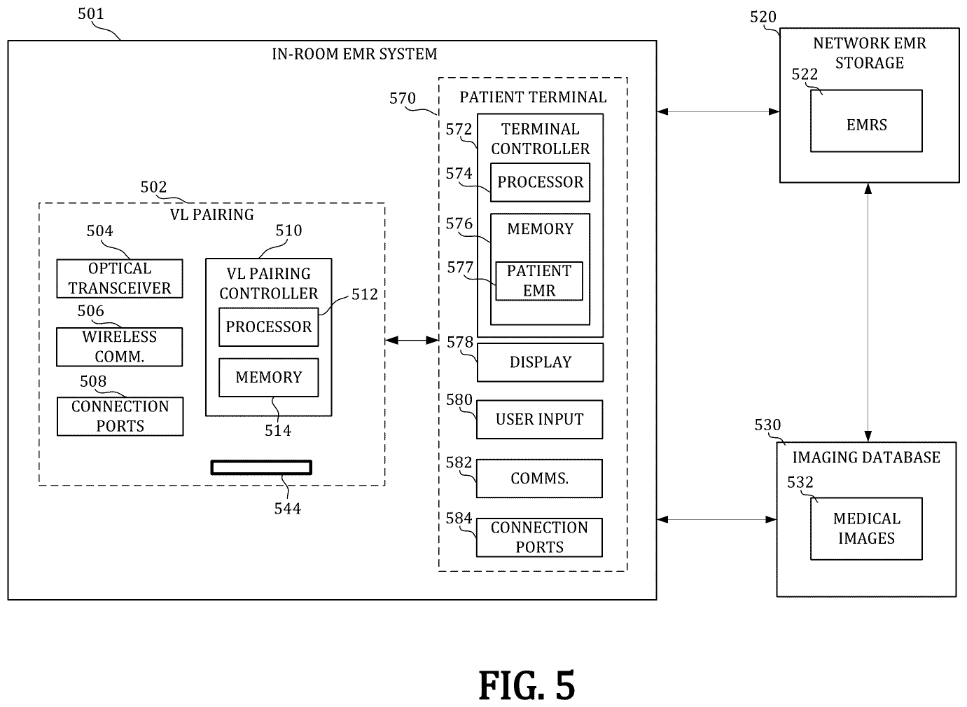

FIG. 5 depicts a schematic diagram of an in-room EMR system 501. The in-room EMR system includes VL-pairing components 502 and a patient terminal 570. The in-room EMR system 501 may be in communication with one or more remote servers, such as a network EMR storage 520 and an imaging database 530 (which may be examples of the remote servers 360 discussed above with respect to FIG. 3).

The components of the VL-pairing components 502 may be incorporated into a stand-alone wireless hub that may be plugged into the patient terminal (such as wireless hub 316 in FIG. 3A). The wireless hub may be a portable, pluggable dongle that includes the VL-pairing components 502. In other examples, the VL-pairing components 502 may be positioned on, or connected to, a circuit board or set of circuit boards (e.g., printed circuit board assembly (PCBA)), and integrated into the patient terminal and/or another component of the anesthesia machine (or other medical device). In examples, the hardware and/or circuitry of the VL-pairing components 502 is integrated and/or non-removable from the medical device. For instance, the components and/or circuit boards may be physically attached inside the housing of the medical device via screws, adhesives, solder, or other attachment means. The components are non-removable in that they are not intended to be removed or detached by a user (with the limited exception of for repair or replacement during maintenance of the medical device).

The VL-pairing components 502 include an optical transceiver 504, a wireless communications device 506 for non-optical communication, and one or more connection ports 508 to interface with the components of the patient terminal or medical device. For instance, the connection ports 508 may include a first connection port and a second connection port. The first connection port may be primarily for streaming video (e.g., a video-out port), such as a DisplayPort or High-Definition Multimedia Interface (HDMI) port. The second connection port may be a data and/or power port, such as a Universal Serial Bus (USB) port.

The material that houses the VL-pairing components 502 (e.g., the housing of the wireless hub) may also include an optical window 544 to allow for the optical signals to reach the optical transceiver 504 and exit the housing. The optical transceiver 504 may be a type of infrared (IR) detector and/or transmitter as described above. The wireless communications device 506 may be the same or similar as wireless communications device 406 described above.

The VL-pairing components 502 also include a pairing controller 510 that includes one or more processors 512 and hardware memory 514. The processor 512 and memory 514 control the pairing process with the video laryngoscope but may not control other operations of the medical device or patient terminal to which the VL-pairing components 502 are attached.

The processor 512 and memory 514, however, may perform additional operations on the received video-laryngoscope data, such as the images and/or video received from the video laryngoscope. For instance, one or more machine learning (ML) models (such as image classifiers) may be stored in the memory 514 of the VL-pairing components 502. The processor 512 may execute one or more of those machine-learning (ML) models with the received video-laryngoscope data as input to the ML model. The output of the model includes additional information or data (e.g., insights) about the video-laryngoscope data that may then be incorporated into the EMR. Because these ML models may be specifically trained for video-laryngoscope data, their inclusion in the VL-pairing components 502 (or the video laryngoscope), such models do not need to be stored or executed on the patient terminal 314, which may be a more general-purpose device.

The patient terminal 570 includes a terminal controller 572 including a processor 574 and memory 576. The patient terminal 570 further includes a display 578, a user input device 580 (e.g., keyboard, mouse, touchscreen), communication devices 582, and connection ports 584. The communication device(s) 582 may provide for Internet or other networked communications, such as to a medical database or monitoring station. The connection ports 584 may receive wired connections. For instance, in some examples where the components of the video laryngoscope VL-pairing components 502 are included as a pluggable module (e.g., wireless hub), the module may be plugged into one of the connection ports 584 of the patient terminal 570.

The processors 512, 574, may be similar to, or the same as processor 412 described above. The processors 512, 574 may perform control, interface, communication, or other processing functions by executing instructions that are stored in the memory 514, 576, respectively. The memory 514, 576 may be similar to, or the same as, memory 414 described above. The memory 514, 576 may store instructions, that when executed by the respective processors 512, 574, cause the respective devices or elements thereof to perform the operations described herein.

The video-laryngoscope data, or a portion thereof, that is received by the VL-pairing components 502 is then transmitted to the patient terminal 570 to be stored in the patient EMR 577 that is being updated or created on the patient terminal 570. The video-laryngoscope data is then displayed by the patient terminal 570 as part of the patient EMR 577 for editing or updating by the user (e.g., the clinician), as discussed further herein.

Once the patient EMR has been updated, edited, and/or confirmed by the user, the patient EMR 577 may be stored on a network EMR storage 520 that stores a plurality of EMRs 522 for multiple different patients. For instance, the updated patient EMR 577 may be transmitted to the network EMR storage 520 for storage with the other EMRs 522. The network EMR storage 520 may be a storage device, such as a server, that is accessible by the patient terminal 570. For instance, the storage device may be a server on the premises of the hospital that includes the patient environment where the patient terminal 570 is placed. In such examples, the patient terminal 570 is accessible via the hospital network. In other examples, the network EMR storage 520 may be a cloud-based device that is accessible via the Internet and is off-premises of the hospital.

In some examples, an imaging database 530, that is separate from the network EMR storage 520 and the patient terminal 570, may be utilized to store larger video or image files. The imaging database 530 may be similar to or the same as a picture archiving and communication system (PACS). The imaging database 530 may be stored on a device stored on the hospital premises and accessible via the hospital network. In other examples, the imaging database 530 may be stored on a cloud-based server that is stored off the hospital premises and accessible via the Internet.

In many examples discussed herein, the imaging data from the video laryngoscope has a sufficiently small size to be stored directly in or with the patient EMR 577. The small size of the video laryngoscope data is primarily due to the intubation procedure lasting only a short duration (e.g., 30 seconds to a few minutes). In some examples, additional video data may be received by the video laryngoscope, such as from an endoscope attached to the video laryngoscope. Such endoscopic video data may be associated with longer endoscopic procedures, and as a result, the endoscopic video data larger than the video laryngoscope imaging data captured by the camera of the video laryngoscope and associated with the intubation procedure itself. That larger video data (e.g., endoscopic video data), however, may still be transmitted from the video laryngoscope to the video laryngoscope VL-pairing components 502 and provided to the patient terminal 570. The patient terminal 570 may then transmit the larger video data to the imaging database 530 to be stored amongst other medical images 532 in the imaging database 530. A link to the larger video data is then returned to the patient terminal 570, and that returned link can be stored within the patient EMR 577 that is being created or updated on the patient terminal 570. When the link is selected, the larger video data is accessed from the imaging database 530.

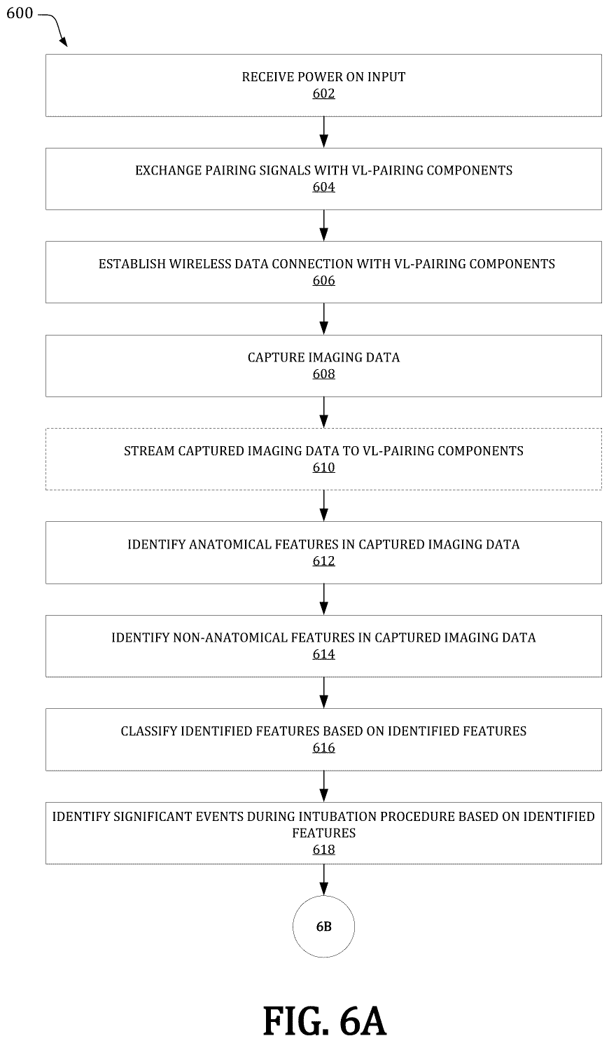

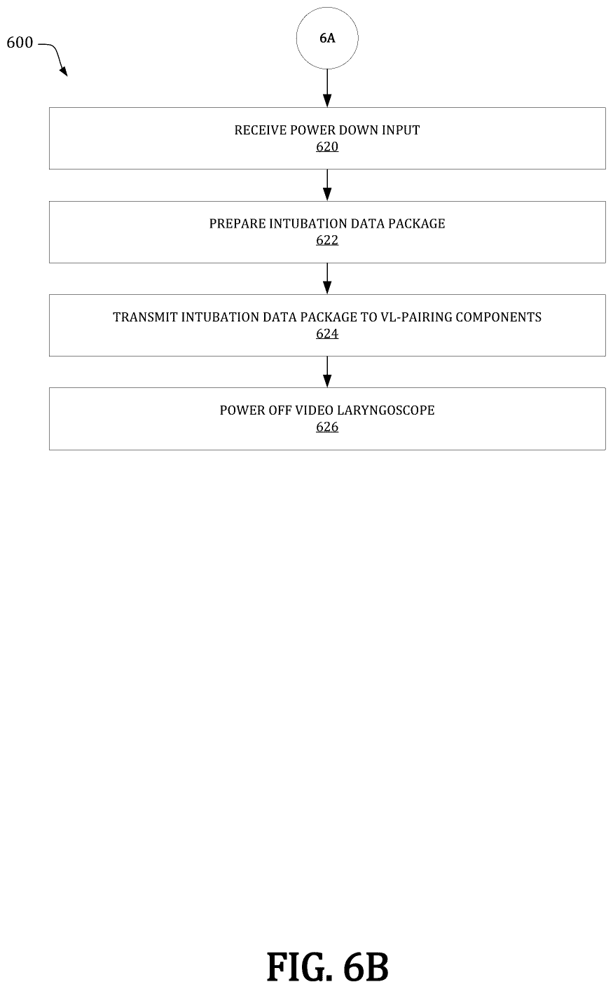

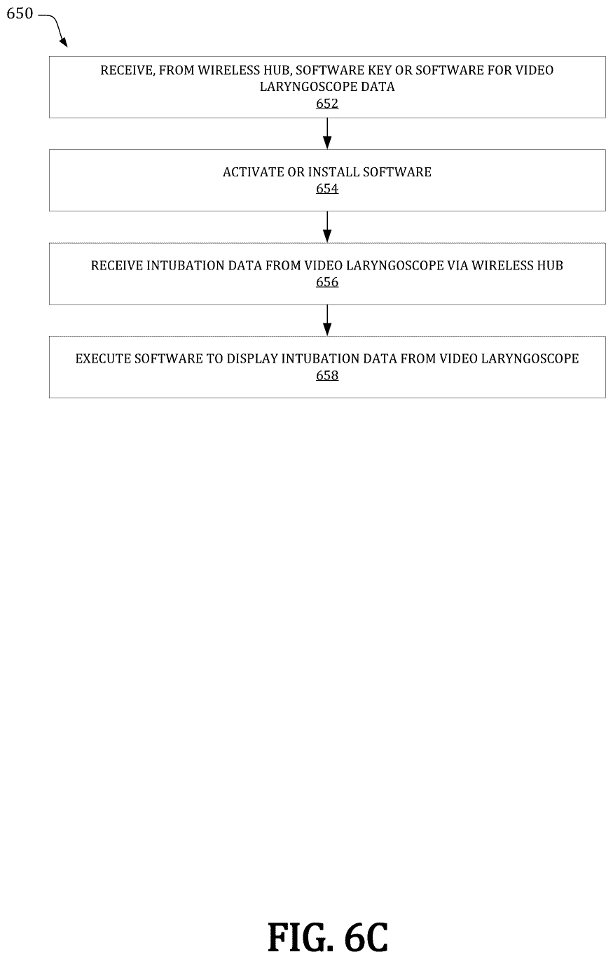

FIGS. 6A-6B depict an example method 600 for generating video-laryngoscope data for integration into an EMR. At operation 602, a power-on input is received by the VL. The power-on input may be a selection of the power button of the video laryngoscope by the user. At operation 604, based on receiving the power-on input, pairing signals are exchanged with the VL-pairing components, such as a wireless hub. The pairing signals may be exchanged without any further user input other than the initial power-on input. Thus, the pairing may occur automatically upon the video laryngoscope being powered on. The video laryngoscope may receive and/or transmit signals with pairing data to the VL-pairing components. The pairing signals may be exchanged via optical signals, such as infrared signals. By having the pairing data be transmitted as an optical signal, there is a greater assurance that the video laryngoscope and the VL-pairing components are located in the same room because the optical signals cannot pass through walls.

At operation 606, a wireless data connection is established. The wireless data connection may be transmitted on a different frequency or signal type than the pairing data in operation 604. For instance, the wireless data connection may be a Bluetooth or WiFi-based connection that allows for video-laryngoscope data to be transmitted from the video laryngoscope to the VL-pairing components.

At operation 608, imaging data is captured by the video laryngoscope. For instance, imaging data is captured by the camera of the video laryngoscope. In some examples, additional video data may be captured from an endoscope attached to the video laryngoscope. At operation 610, the captured imaging data may be streamed to the VL-pairing components in substantially real time (e.g., with less than a 1 second delay). The device (e.g., patient terminal, monitor) attached to the VL-pairing components may then display the captured imaging data.

At operation 612, anatomical features are identified in the captured imaging data. The anatomical features may include different part of anatomy of the patient, such as the vocal cords. The identification of the anatomical features may be performed through the use of computer vision techniques, which may include the use of trained ML models, such as convolutional neural networks (CNNs), that are stored and executed on the video laryngoscope. For instance, the ML model may be trained for particular types of object recognition, such as the anatomical features. The detection of the anatomical features may be performed as the images are being captured and/or on a frame-by-frame basis. In other examples, the identification of the anatomical features may be performed at a delayed rate and/or after the intubation procedure has been completed. The identified anatomical features may be stored along with the frame and/or timestamp for which the anatomical feature was identified. For instance, a data item may be created for each frame in which vocal cords are identified.

At operation 614, non-anatomical features are identified in the captured imaging data. The non-anatomical features may include features such as the ETT and/or other tools (e.g., stylet, bougie, introducer, endoscope). The identification of the non-anatomical features may be performed through the use of trained ML models, such as convolutional neural networks CNNs, that are stored and executed on the video laryngoscope. For instance, the ML model may be trained for particular types of object recognition, such as the non-anatomical features. The identified non-anatomical features may be stored along with the frame and/or timestamp for which the non-anatomical feature was identified. For instance, a data item may be created for each frame in which the ETT is identified. While operations 612, 614 are depicted as separate operations, they may be performed at substantially the same time or in a different order. In some examples, the identification of multiple different anatomical and/or non-anatomical features may be performed with the same ML model(s). The non-anatomical features may also include subparts or portions of an instrument or tube, such as the distal tip of the ETT.

At operation 616, one or more of the identified features in operations 612, 614 are classified. The classification may include grading the particular feature that was identified. For instance, for the identified vocal cords, a best or most complete view of the vocal cords may be identified. This classification may be performed in some examples by the same ML model that identified the feature. In other examples, additional image analysis algorithms, heuristics, or ML models may be applied that classify the features.

Other classifications may include a grade of the airway. This grading of the airway may be based on the identified anatomical features, such as the vocal cords. For instance, the airway grade may be based on an amount of the vocal cords that can be seen in an image and/or a percentage of the glottic opening, among other types of grading factors. Determining such an airway grade may include identifying a frame from the captured imaging data that best shows the vocal cords, and then generating the airway grade from that identified image.

The classifications may also be related to the instruments used during the procedure, such as the ETT tube itself and/or features of the laryngoscope. For instance, the ETT size (e.g., diameter) may be classified from the image frames that include the ETT. The insertion depth of the ETT may also be classified based on a detection of the depth markings on the outside of the ETT. The insertion depth may be determined for each frame for which the depth markings can be seen in the frame, and/or a maximum insertion depth for the intubation procedure may be determined based on a comparison of the insertion depths for each frame.

The classifications may also be based on multiple factors and/or other classifications that have been performed. For example, an intubation-difficulty grade may be classified as part of operation 616. The intubation-difficulty grade may be based on the length of the intubation procedure, detected trauma in the airway, and/or multiple attempts at inserting the ETT through the vocal cords, among other factors. In some examples, the video laryngoscope may also receive data from a patient monitor or other sensors, such as a pulse oximeter, which can provide data about desaturation (e.g., reduction in SpO2 values). Such sensor data (e.g., pulse oximetry data) may be used in generating the intubation difficulty grade. Of note, the intubation-difficulty grade may be different from the airway grade. For instance, in some cases, a high airway grade may still lead to a difficult intubation, and a low airway grade may lead to a relatively easy intubation. Thus, the intubation-difficulty grade may be of particular interest for clinicians for future intubations of the patient. For example, a high airway grade (indicating a likely routine intubation) with a difficult intubation grade is unexpected and provides unique insight to clinicians performing future intubations. Such information may also be useful for clinicians performing the extubation procedure because difficult intubations often result in difficult extubations.

At operation 618, significant events during the intubation procedure are identified based on the features identified in operations 612, 614 and/or the classifications determined in operation 618. The significant events may include identification of the vocal cords and/or when the best (e.g., most complete) view of the vocal cords occurred prior to the ETT being inserted through the vocal cords. Another significant event may be the first detection of the tip of the ETT and/or the detection of any other instruments or other non-anatomical features. Yet another significant event may be when the ETT is fully inserted through the vocal cords. These significant events may be used to extract and display frames from the imaging data that correspond to the significant events. The significant events may also be marked in a media playback interface to allow for advancing the video playback to the particular events.

At operation 620, a power-down input is received. The input may be a selection of the power button to power down the video laryngoscope. Based on receiving the power-down input, the video laryngoscope performs additional operations and transmissions prior to actually powering off the video laryngoscope. For instance, based on receiving the power-down input, an intubation data package is prepared at operation 622. The intubation data package includes the imaging data captured throughout the intubation procedure along with metadata and/or text based on the detected features, classifications, and/or significant events determined or identified during method 600. For instance, the detected features, classifications, and/or significant events may be stored as metadata of the video file corresponding to the captured imaging data. These metadata values may be stored as defined variables with the values for the variables corresponding to the detected or identified features, classifications, and/or events determined through method 600. The intubation data package may further include data regarding the duration of the intubation procedure and/or the location of the video laryngoscope during the intubation procedure (e.g., as identified from the exchange of data with the locating beacon). The intubation data package may also include a number of intubation attempts that were performed. For instance, the video data from the video laryngoscope may be analyzed to identify the number of attempts that were performed.

At operation 624, the intubation data package is transmitted to the VL-pairing components (e.g., the wireless hub). Subsequent to the transmission of the intubation data package, the video laryngoscope is powered off at operation 626. Accordingly, in some examples, the only user interaction that is needed is a powering on of the video laryngoscope. Once the power-on input is received, the video laryngoscope automatically pairs with the VL-pairing components and further automatically prepares and transmits the intubation data package prior to powering off. As a result, the clinician is able to more directly focus on the patient and intubation procedure, rather than focusing on interacting with a user interface of the video laryngoscope.

Also of note, the streaming of the captured imaging data and handling of the intubation data package may be performed by the same VL-pairing components (e.g., wireless hub). For example, a single wireless hub may provide the streamed image content to a device for display during the intubation procedure and then also pass the intubation data package (once received) to a device (e.g., patient terminal) for incorporation into an EMR. In some examples, the wireless hub may be moved between devices for the two different functions. As an example, the wireless hub may be plugged into a monitor during the intubation procedure to allow for streaming to the monitor and then plugged into the patient terminal for transmission of the intubation data package.

In some examples, the different ports of the wireless hub may serve different purposes to allow for streaming and/or transfer of the intubation data package. For instance, a video-out port (e.g., HDMI port) of the wireless hub may be connected to a first device (e.g., monitor) and a data and/or power port (e.g., USB port) may be connected to a second device (e.g., patient terminal). Thus, streaming of the captured image data may be facilitated by the first connection port, and powering of the wireless hub and transmission of the intubation data package may be accomplished by the second connection port.

Once the VL-pairing components receive the intubation data package, the VL-pairing components may store the intubation data package in memory and/or further transmit the intubation data package to a separate device, such as the patient terminal. For instance, when the intubation data package is received from the VL, the wireless hub may not be currently connected to the patient monitor. Nevertheless, the intubation data package may be stored in the memory of the wireless hub. When the wireless hub is then connected to the patient terminal, the stored intubation data package is then transferred from the wireless hub to the patient terminal.

While the determinations and image analysis operations of method 600 are described above as being performed by the video laryngoscope, in some examples, one or more of the operations may be performed by the wireless hub (e.g., the VL-pairing components). For example, one or more of the identifications or classifications of operations 612, 614, 616, and 618 may be performed by the wireless hub. As an example, the wireless hub may store and execute the corresponding ML model that performs the identifications and/or classifications. In some examples, the intubation data package may alternatively be generated on the wireless hub. For example, the captured image data may be transmitted from the video laryngoscope to the wireless hub for further processing and creation of the intubation data package that is ultimately provided to the patient terminal.

In some examples, the patient terminal, MPM, and/or anesthesia machine may not have software, or may not need to constantly run software, capable of displaying video laryngoscope data, such as video from the video laryngoscope that is streamed to the device and/or the intubation data package. For instance, the patient terminal, MPM, and/or anesthesia machine may have software already installed that allows for the video laryngoscope data to be processed. That software, however, may not need to be active until a video laryngoscope is coupled to the respective device. In other examples, the device (e.g., patient terminal, MPM, and/or anesthesia machine) may not have the software installed at all. With the present technology, the software can be provided by the wireless hub and/or activated by the wireless hub.

FIG. 6C depicts an example method 650 for installing or activating software for viewing intubation data. The example method 650 may be performed by an anesthesia machine, an MPM, and or a patient monitor, among other devices.

At operation 652, a software key or software itself (e.g. a software program or an update to a software program) for processing and displaying intubation data, from a video laryngoscope, is received from a wireless hub (e.g., a pluggable dongle with VL-pairing components). For instance, the anesthesia machine, MPM, patient terminal, or other device (e.g., computing device or device having computing capabilities), receives the software key or software when the wireless hub is plugged into the anesthesia machine, MPM, patient terminal, or other device.

At operation 654, the software is activated or installed. For example, when the software key is received and the software was previously installed, the software key is used to activate software. The software key may be a product key, license key, or activation key. The software key may be a unique string of characters (e.g., cryptographic token) that validates and/or activates the software installed on the device. In some examples, the software is encrypted or otherwise locked until the software key is received. In other examples, the software is partially unlocked (e.g., a subset of features are available) without the software key, and the full set (or a larger set of features) of software are enabled when the software key is received. Once the key is received form the wireless hub, the software may execute an algorithm to verify the key (e.g., verifying format and/or checksum of the key) or use a network-based verification. Based on verification of the key, the software (or additional features of the software) are activated.

In examples where the software (or updates to the software) is received in operation 654, the device installs the software (or updates). The device is then ready to execute the software to process and display the intubation data received from the video laryngoscope. By providing the software and/or updates from the wireless hub, software may be more easily distributed and updated to the various medical devices that require the software. Because the wireless hub is portable, it may be used for multiple devices at different times. In addition, the wireless hub may be loaded with updates or new software more easily, due to its portability, than large medical devices, such as anesthesia machines.

At operation 656, intubation data generated by the video laryngoscope is received via the wireless hub plugged into the anesthesia machine, MPM, patient terminal, or other device. The intubation data may include video data captured by the video laryngoscope and/or other types of data within an intubation data package, as discussed in further detail herein.

At operation 658, the software is executed to cause the processing and/or display of the intubation data received from the video laryngoscope. For instance, the anesthesia machine, MPM, patient terminal, or other device executes the software and provides the received intubation data as input. The software processes the intubation data and causes the intubation data (or a portion thereof) to be displayed on a display of the device. The software may also provide additional features for searching or parsing the intubation data and/or algorithms that identify relevant portions of the intubation data to populate an EMR. In some examples, the software may include the ML models and/or analysis algorithms to perform the determinations and identifications of operations 612-618 of method 600. In such examples, the device executing the software may perform the determinations and identifications alternatively, or in addition to, to the video laryngoscope and/or wireless hub.

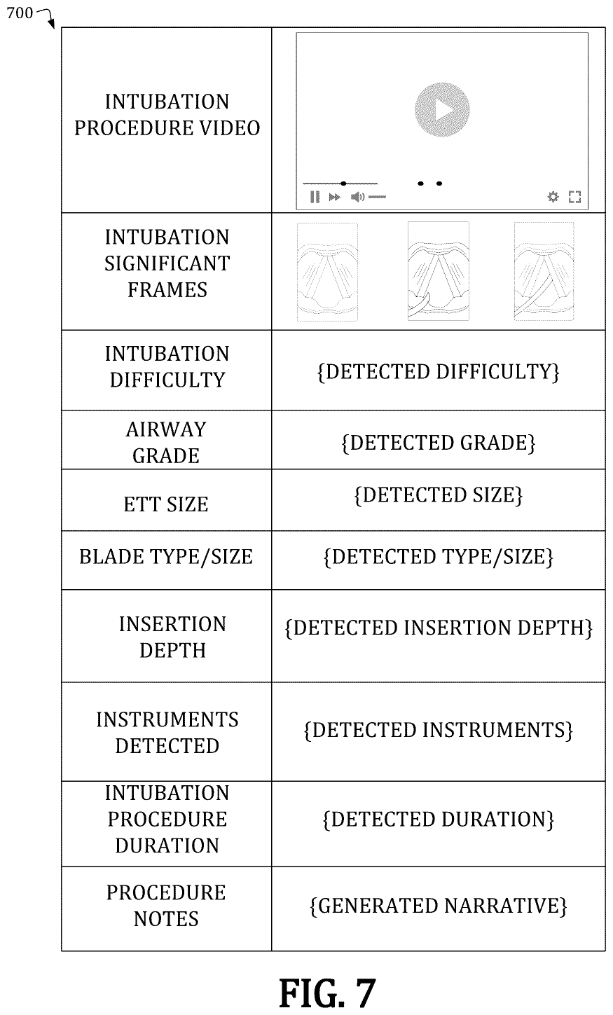

FIG. 7 depicts example segments of an EMR for intubation data. The example segments are depicted as a table 700 in FIG. 7, but the intubation data may be displayed in different formats in other examples. The table 700 includes multiple segments that may be displayed on the patient terminal for interaction with the clinician and/or verification by the clinician and incorporation into the patient EMR.

The different categories of intubation data may be listed in a first column of the table, and the determined values for those different categories (as included in the intubation data package) are provided in the corresponding second column. In the example depicted, the categories of intubation data include the intubation procedure video, intubation significant frames, intubation difficulty, airway grade, ETT size, laryngoscope blade type/size, insertion depth, instruments detected, intubation procedure duration, and procedure notes. In other examples, there may be more or fewer categories of intubation data.

The intubation procedure video may be populated with a media player for playing a video of the captured images rom the intubation procedure. Particular points may be indicated in the progress bar or playback bar (indicated by black dots in FIG. 7). The particular points correspond to the significant events identified in the captured image data. Text labels may also be added to the indicated points to indicate the significant event to which the particular point corresponds.

The intubation significant frames may include multiple, individual frames from the captured images that correspond to the identified significant events. In the example depicted, a first frame is shown for a view of the vocal cords prior to the ETT being detected, a second frame is shown for a view of the vocal cords when the ETT tip is detected, and a third frame is shown for a view when the ETT tip is fully inserted through the vocal cords.

The intubation difficulty and the airway grade are populated with the respective determined difficulty and grade. The ETT size, blade type/size, insertion depth, instruments detected, and intubation procedure duration category are also populated with respective determinations from the video laryngoscope. The fields may be populated with text and/or a representative frame from the image data that supports the determination.

The procedure-notes category may be populated with an automatically generated natural language narrative that describes the intubation procedure. The natural language narrative may be generated by an ML model, such as a transformer network, an adversarial neural network system, and/or a generative artificial intelligence (AI) model. The ML model may be one that is multi-modal in that that the model can accept multiple modalities as an input, such as text and images. The input to the ML model may then include one or more image frames from the captured images. Alternatively or additionally, the input to the ML model may include one or more of the values determined in method 600, such as the determine intubation difficulty, airway grade, ETT size, blade type/size, insertion depth, instruments detected, and/or intubation procedure duration, among other information. The ML model then processes the input and generates the narrative for the procedure notes as output from the model. In some models, one or more of the asserted facts in the narrative, a citation to the particular data item from the input (e.g., particular category of metadata) supports the asserted fact. The citation may also include a frame and/or link to a video segment that supports the asserted fact.

The intubation data package may also include any key moments or still screenshots that were manually captured by the clinician during the intubation procedure. For instance, during the intubation procedure, the clinician may mark key events and/or capture still images (e.g., screenshots), which may be included in the video data.

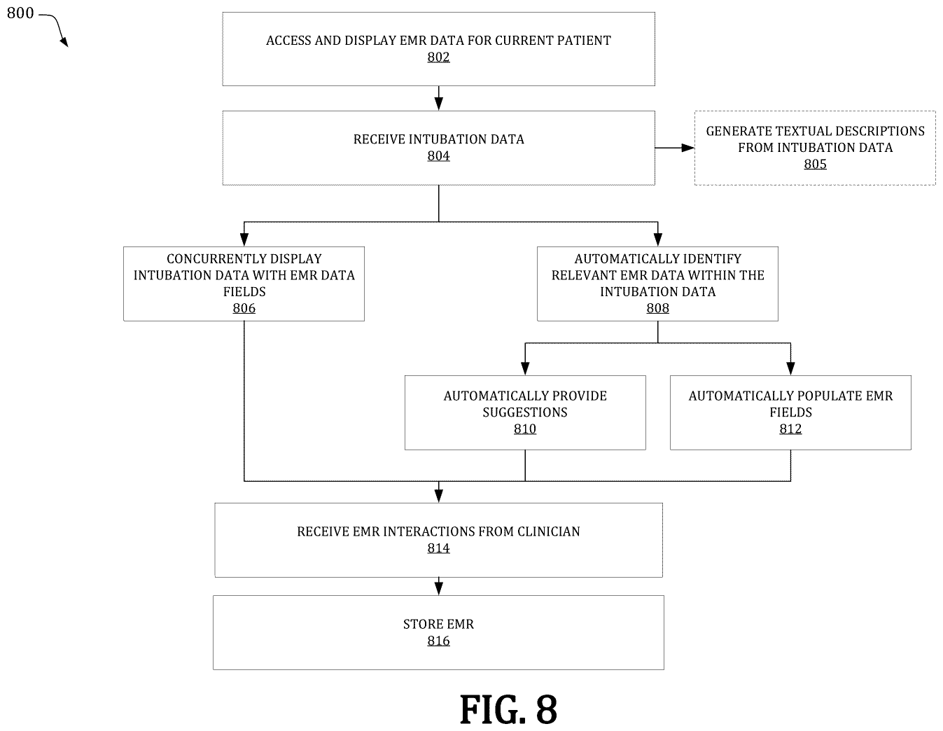

FIG. 8 depicts a method 800 for populating an electronic medical record with data associated with intubation procedure. The operations of method 800 may be performed by the patient terminal and/or other suitable computing components for populating an EMR.

At operation 802, EMR data for the current patient is accessed and displayed. Accessing the EMR data may include accessing a remote database storing the EMR data for the patient, such as through a web portal or specialized application operating on the patient terminal. For instance, a clinician may need to input particular credentials to gain access to the database and provide identifying information about the patient to retrieve the EMR for the patient. The access may be performed over a network connection, such as a hospital network connection. The connection may provide access to databases or servers that are located on premises of the hospital and/or cloud-based servers accessible via the Internet.

With the patient EMR accessed, the intubation data package is received from the VL-pairing components in operation 804. In addition to the intubation data package from the video laryngoscope, additional monitored parameters for the patient may be received from MPM that may also be used in populating the EMR. In some examples, the video data may include still screenshots from the video feed(s). For instance, during the intubation procedure, the clinician may capture still images (e.g., screenshots), which may be included in the video data. In other examples, the video data may also include objects that were identified within the video feed(s) (e.g., ETT, mask, vocal cords) and time stamps at which such objects were identified. For instance, upon objects and/or events being identified in the video feed, screenshots of those images or shortened portions of the video surrounding the events (e.g., 5-10 second clips) may be identified or included in the video data. The video data may also include additional data extracted from the video feed(s), as discussed above. For instance, the computer vision technology discussed herein may be able to further classify the particular ETT based on its size. As a result, the size of the ETT can automatically be identified from the video data. The type and/or size of the laryngoscope blade used on the video laryngoscope may also be able to be automatically identified from the video data. In addition, the video data may be analyzed to generate a difficulty score for the intubation procedure. The video data may also be analyzed to identify any anatomical anomalies, such as lesions, discolorations, or other abnormalities. For instance, the coloring of the anatomy may be analyzed to determine departures from expected coloring, such as green vocal cords. Such detected anomalies and/or abnormalities may be included in the intubation data package and used to populate the EMR. In other examples, the patient terminal and/or the wireless hub may perform the computer vision and/or algorithmic procedures to extract the additional data from the captured image data (e.g., video feed) from the video laryngoscope.

With respect to the patient parameters that may also be received from the MPM and/or other sensors, all the available patient parameter data recorded during the intubation procedure may be included with the intubation data that is used to populate the EMR. In other examples, only the patient parameter data displayed during the intubation procedure is included in with intubation data. In some examples, the patient parameter data is further analyzed to extract minimums, maximums, or other anomalies, such as a lowest SpO2 level and/or a blood pressure spike.

By receiving the intubation data while the patient-specific EMR is accessed and open on the patient terminal, the intubation data is automatically implicitly associated with the patient undergoing the intubation procedure without having to tag the intubation data itself. For instance, by incorporating the intubation data into the patient's EMR during the surgical procedure, the intubation data itself does not need to be stored separately and identified with the patient. Such an enhancement reduces potential privacy risks and significantly reduces storage and data management requirements for the patient's personal health data.

The intubation data may be provided through an AIMS interface or similar interface. The portions of the intubation may be received from the anesthesia machine, such as from the MPM section of the anesthesia machine. In other examples where the video laryngoscope wireless hub is plugged into the patient terminal directly, the video laryngoscope video data may be received via the wireless module rather than from the anesthesia machine or MPM section thereof.

At optional operation 805, one or more textual descriptions may be generated from the intubation data, such as a natural language description for notes or a summary regarding the intubation procedure. As discussed above, the natural language narrative may be generated by an ML model, such as a transformer network, an adversarial neural network system, and/or a generative AI model. Accordingly, at operation 805, the patient terminal may provide the intubation data, or a portion thereof, as input to the ML model. The intubation data may include all data, or a subset of data, from the intubation data packages as well as one or more patient parameters received from the MPM or other sensors. Providing the input may include a local transmission for an ML model that operates on the patient terminal and/or transmitting the input to a remote ML model (such as a cloud-based generative AI model). The ML model then processes the input and generates the textual description(s), such as the narrative summary or notes for the intubation procedure. The patient terminal then receives that output, and may then display the textual with, or incorporate into, the EMR as part of the intubation data.

Once the intubation data is received, operation 806 and/or operation 808 may be performed. In operation 806, the intubation data is displayed with the fillable fields of the EMR for the patient. In some examples, the intubation data is displayed concurrently with the fillable fields on the patient terminal. Accordingly, the clinician can more quickly and accurately populate the fillable fields. The display of the intubation field may include video player with the video data from the video laryngoscope. The clinician may be able to navigate through the video to identify particular events or features that are desired to be reviewed by the clinician. Captured still screenshots may also be displayed along with other data captured by the classifications of the objects within the video feed(s). In some examples, the table 700 (or segments thereof) may be displayed by the patient terminal either as part of the EMR and/or concurrently with the EMR to assist in filling of the EMR.

At operation 808, data within the intubation data that is relevant to the fillable fields of the EMR data is automatically identified. For instance, for a topic of a fillable field, a search query may be executed over the intubation data to identify the data that is relevant to the particular field. If the computer vision techniques to extract data from the video feed(s) have not been previously performed, such techniques may be performed at operation 808 and targeted to extract data specific to the particular fields of the EMR for the patient.

At operation 810, based on the relevant data identified in operation 808, suggestions for one or more of the fillable fields of the EMR are presented. For example, suggested entries based on the intubation data may be displayed adjacent to one or more fillable fields for which relevant data was identified. A clinician may then interact with the suggested data to cause the data to be populated in the respective field. At operation 812, the fillable fields for which relevant intubation data was identified are automatically populated with identified relevant data. Such automatic populations or suggestions in operations 810 and/or 812 greatly reduce the number of inputs that the patient terminal needs to process from the clinician and also reduces the total time required from the clinician to complete the EMR for the patient before being able to complete additional medical procedures.

At operation 814, interactions with the patient terminal are received. The interactions may be to simply accept the automatically populated data in the EMR. In other examples, the interactions may edit or adjust the data within the EMR. The interactions may also further augment the EMR data, such as by adding notes or other narratives to the EMR for the patient. The clinician may then accept and submit the EMR for storage.

At operation 816, once the clinician has edited and/or accepted the entries within the EMR, the updated EMR is stored. The EMR may be stored locally on the patient terminal and/or uploaded to a database on a hospital server or cloud-based server for storage and later remote access. The EMR may include not only the intubation data that was incorporated into the fillable fields of the EMR, but the EMR may also include the intubation data itself, such as a copy of the video file(s) from the video laryngoscope and/or any metadata included in the intubation data package.

Once the EMR is saved and stored, the intubation data stored in other locations may be deleted. For example, once the intubation data has been stored in the EMR (or at least the relevant portions thereof), the intubation data may be deleted from the other memory, such as memory of the wireless hub, the MPM, and/or the video laryngoscope. For instance, a signal may also be transmitted back to the video laryngoscope to indicate that the corresponding video file can be deleted from the memory of the video laryngoscope. The video laryngoscope may then delete the corresponding video file from its memory.

After the EMR has been stored, the EMR can then be later accessed by clinicians when performing a subsequent procedure, such as another intubation procedure or an extubation procedure. The intubation data segments of the EMR provide valuable insights to the clinician for performing such procedures. For example, indications of one difficult intubation likely indicate that future intubations, and the subsequent extubation, are likely to be difficult. The clinician may then review the video file, captured images, and/or other data to make informed decisions about to perform the upcoming procedure. The intubation within the EMR may also serve as a valuable resource for clinicians in the intensive care unit (ICU) that is treating the patient immediately (e.g., same day or next day) after the intubation procedure was performed. Ear, Nose, and Throat (ENT) clinicians may also find such intubation data within the EMR to be particularly useful for later consults.

While the foregoing discussion has been primarily related to video laryngoscope data, the above techniques and processes may be similarly applied to internal video data captured by other video-capture internal imaging devices that are at least partially inserted into the patient. Such captured video may be analyzed to extract the features and properties discussed above and incorporated into the EMR in a similar fashion via the internal imaging device.

As should be appreciated from the foregoing, aspects of the technology relate to incorporating video laryngoscope data into an EMR. In an aspect, the technology relates to a computer-implemented method for incorporating intubation data into an electronic medical record of a patient. The computer-implemented method also includes accessing the electronic medical record of the patient. The method also includes receiving, from wireless hub wirelessly coupled to a video laryngoscope, intubation data generated by the video laryngoscope during an intubation procedure of a patient, where the intubation data includes video data captured by the video laryngoscope and metadata for the video data generated by the video laryngoscope. The method also includes concurrently displaying at least a portion of the intubation data with fillable fields of the electronic medical record. The method also includes receiving at least one interaction with the electronic medical record during the concurrent display. The method also includes storing the electronic medical record based on the at least one interaction.

Examples may include one or more of the following features. The computer-implemented method where concurrently displaying at least the portion of the intubation data includes displaying a video player with the video data. The computer-implemented method may include executing a query over the intubation data to identify intubation data relevant to at least one of the fillable fields of the electronic medical record. The computer-implemented method may include displaying one or more suggested entries to one or more of the fillable fields based on the identified relevant intubation data. The computer-implemented method may include automatically filling one or more of the fillable fields with the identified relevant intubation data. Storing the electronic medical record includes storing the video data with the electronic medical record. The metadata includes at least one of an airway grade or an intubation-difficulty grade. The metadata includes at least one of an endotracheal tube size or a laryngoscope blade type or size. The metadata includes at least one of intubation procedure duration or a number of intubation attempts. The metadata includes an insertion depth of an endotracheal tube or an indication of one or more instruments detected in the video data.

In another aspect, the technology relates to a video laryngoscope. The video laryngoscope also includes a body; a display screen, a laryngoscope camera coupled to a distal end of the body, a processor. The laryngoscope also includes memory storing instructions that, when executed by the processor causes the video laryngoscope to perform operations may include: establish a wireless data connection with a wireless hub coupled to a patient terminal; capture, by the laryngoscope camera, image data during an intubation procedure; identify one or more anatomical features within the captured image data; identify one or more non-anatomical features within the captured image data; generating metadata, for the image data, based on the identified one or more anatomical features and the one or more non-anatomical features; generating an intubation data package may include the image data and the metadata; and transmitting, via the wireless data connection, the intubation data package to the wireless hub for incorporation into a patient electronic medical record at the patient terminal.

Examples may include one or more of the following features. The video laryngoscope where the one or more anatomical features may include vocal cords. The one or more non-anatomical features may include an endotracheal tube. Establishing the wireless data connection with the wireless hub may include: in response to receiving a power-on input, exchanging optical pairing signals via optical signals with the wireless hub; and establishing the wireless data connection based on the pairing signals, where the wireless data connection is a non-optical connection. The operations further may include: receive a power-down input; where the transmitting of the intubation data packages is performed subsequent to receiving the power-down input and prior to the video laryngoscope powering off. The operations further may include: classify one or more of the anatomical features or the non-anatomical features; where the classifications of the one or more of the anatomical features or the non-anatomical features is included in the metadata. The operations further may include: based on the one or more of the identified anatomical features or the non-anatomical features, identifying one or more significant events during the intubation procedure; where the identified one or more significant events are included in the metadata.

In another aspect, the technology relates to an intubation system the includes a video laryngoscope and a wireless hub. The video laryngoscope includes a laryngoscope camera; a first processor; and first memory storing instructions that, when executed by the first processor causes the video laryngoscope to perform operations. The operations include capture, by the laryngoscope camera, image data during an intubation procedure; identify one or more anatomical features or non-anatomical features within the captured image data; generate metadata, for the image data, based on the identified one or more anatomical features or non-anatomical features; and generate an intubation data package comprising the image data and the metadata. The wireless hub is in wireless communication with the video laryngoscope. The wireless hub includes a port coupled to a patient terminal; a second processor; and second memory storing instructions that, when executed by the second processor causes the wireless hub to perform operations. The operations include receive, from the video laryngoscope, the intubation data package; and transmitting, via the port, the intubation data package to the patient terminal.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A computer-implemented method for incorporating intubation data into an electronic medical record of a patient, the method comprising:
accessing the electronic medical record of the patient;
receiving, from a wireless hub wirelessly coupled to a video laryngoscope, intubation data generated by the video laryngoscope during an intubation procedure of a patient, wherein the intubation data includes video data captured by the video laryngoscope and metadata for the video data generated by the video laryngoscope;
concurrently displaying at least a portion of the intubation data with fillable fields of the electronic medical record;
receiving at least one interaction with the electronic medical record during the concurrent display; and
storing the electronic medical record based on the at least one interaction.

2. The computer-implemented method of claim 1, wherein concurrently displaying at least the portion of the intubation data includes displaying a video player with the video data.

3. The computer-implemented method of claim 1, further comprising executing a query over the intubation data to identify intubation data relevant to at least one of the fillable fields of the electronic medical record.

4. The computer-implemented method of claim 3, further comprising displaying one or more suggested entries to one or more of the fillable fields based on the identified relevant intubation data.

5. The computer-implemented method of claim 3, further comprising automatically filling one or more of the fillable fields with the identified relevant intubation data.

6. The computer-implemented method of claim 1, wherein storing the electronic medical record includes storing the video data with the electronic medical record.

7. The computer-implemented method of claim 1, wherein the metadata includes at least one of an airway grade or an intubation-difficulty grade.

8. The computer-implemented method of claim 1, wherein the metadata includes at least one of an endotracheal tube size or a laryngoscope blade type or size.

9. The computer-implemented method of claim 1, wherein the metadata includes at least one of intubation procedure duration or a number of intubation attempts.

10. The computer-implemented method of claim 1, wherein the metadata includes an insertion depth of an endotracheal tube or an indication of one or more instruments detected in the video data.

11. A video laryngoscope, comprising:
a body;
a display screen;
a laryngoscope camera coupled to a distal end of the body;
a processor; and
memory storing instructions that, when executed by the processor causes the video laryngoscope to perform operations comprising:
establish a wireless data connection with a wireless hub coupled to a patient terminal;
capture, by the laryngoscope camera, image data during an intubation procedure;
identify one or more anatomical features within the captured image data;
identify one or more non-anatomical features within the captured image data;
generating metadata, for the image data, based on the identified one or more anatomical features and the one or more non-anatomical features;
generating an intubation data package comprising the image data and the metadata; and
transmitting, via the wireless data connection, the intubation data package to the wireless hub for incorporation into a patient electronic medical record at the patient terminal.

12. The video laryngoscope of claim 11, wherein the one or more anatomical features comprise vocal cords.

13. The video laryngoscope of claim 11, wherein the one or more non-anatomical features comprise an endotracheal tube.

14. The video laryngoscope of claim 11, wherein establishing the wireless data connection with the wireless hub comprising:
in response to receiving a power-on input, exchanging optical pairing signals via optical signals with the wireless hub; and
establishing the wireless data connection based on the pairing signals, wherein the wireless data connection is a non-optical connection.

15. The video laryngoscope of claim 11, wherein the operations further comprise:
receive a power-down input;
wherein the transmitting of the intubation data packages is performed subsequent to receiving the power-down input and prior to the video laryngoscope powering off.

16. The video laryngoscope of claim 11, wherein the operations further comprise:
classify one or more of the anatomical features or the non-anatomical features;
wherein the classifications of the one or more of the anatomical features or the non-anatomical features is included in the metadata.

17. The video laryngoscope of claim 11, wherein the operations further comprise:
based on the one or more of the identified anatomical features or the non-anatomical features, identifying one or more significant events during the intubation procedure;
wherein the identified one or more significant events are included in the metadata.

18. An intubation system, comprising:
a video laryngoscope, comprising:
a laryngoscope camera;
a first processor; and
first memory storing instructions that, when executed by the first processor causes the video laryngoscope to perform operations comprising:
capture, by the laryngoscope camera, image data during an intubation procedure;
identify one or more anatomical features or non-anatomical features within the captured image data;
generate metadata, for the image data, based on the identified one or more anatomical features or non-anatomical features; and
generate an intubation data package comprising the image data and the metadata; and
a wireless hub, in wireless communication with the video laryngoscope, the wireless hub comprising:
a port coupled to a patient terminal;
a second processor; and
second memory storing instructions that, when executed by the second processor causes the wireless hub to perform operations comprising:
receive, from the video laryngoscope, the intubation data package; and
transmit, via the port, the intubation data package to the patient terminal.

19. The intubation system of claim 18, further comprising the patient terminal comprising:
a third processor; and
third memory storing instructions that, when executed by the third processor, cause the patient terminal to perform operations:
access an electronic medical record of the patient;
receive, from the wireless hub, the intubation data package;
concurrently display at least a portion of the intubation data with fillable fields of the electronic medical record;
receive at least one interaction with the electronic medical record during the concurrent display; and
store the electronic medical record based on the at least one interaction.

20. The intubation system of claim 18, wherein the metadata includes at least two of significant frames from the captured image data, intubation difficulty grade, airway grade, endotracheal tube size, endotracheal tube insertion depth, number of intubation attempts, or intubation procedure duration.
